(19) United States
(12) Patent Application Publication
(10) Pub. No.: US 2023/0225659 A1
(43) Pub. Date: Jul. 20, 2023

(54) Biosignal Sensing Device Using Dynamic Selection of Electrodes

(71) Applicant: Apple Inc., Cupertino, CA (US)

(72) Inventors: Erdrin Azemi, San Mateo, CA (US); Ali Moin, San Mateo, CA (US); Anuranjini Pragada, Cupertino, CA (US); Jean Hsiang-Chun Lu, Sunnyvale, CA (US); Victoria M. Powell, San Francisco, CA (US); Juri Minxha, Seattle, WA (US); Steven P. Hotelling, Los Gatos, CA (US)

(21) Appl. No.: 18/094,841

(22) Filed: Jan. 9, 2023

Related U.S. Application Data
(60) Provisional application No. 63/299,864, filed on Jan. 14, 2022.

(51) Int. Cl. A61B 5/304; A61B 5/291; A61B 5/00; A61B 5/31

(52) U.S. Cl. CPC A61B 5/304; A61B 5/291; A61B 5/6817; A61B 5/31; A61B 2562/0209; A61B 2562/046

(57) ABSTRACT

A wearable electronic device includes a housing, and an electrode carrier attached to the housing and having a nonplanar surface. The wearable electronic device includes a set of electrodes, including electrodes positioned at different locations on the nonplanar surface. The wearable electronic device includes a sensor circuit and a switching circuit. The switching circuit is operable to electrically connect a number of different subsets of one or more electrodes in the set of electrodes to the sensor circuit.

### CROSS-REFERENCE TO RELATED APPLICATION

This application is a nonprovisional and claims the benefit under 35 U.S.C. § 119(e) of U.S. Provisional Patent Application No. 63/299,864, filed Jan. 14, 2022, the contents of which are incorporated herein by reference as if fully disclosed herein.

### TECHNICAL FIELD

Embodiments described herein generally relate to a wearable electronic device for measuring biological signal parameters from a user.

### BACKGROUND

Brain activity can be monitored using electrodes placed on the scalp of a user. The electrodes may in some cases be placed inside or around the outer ear of the user. Measuring of the brain activity using electrodes placed in or around the outer ear may be preferred due to benefits such as reduced device mobility and decreased visibility of the electrodes when compared to other devices that require electrodes to be placed on visible areas around the scalp of the user. However, for accurate measurements of brain activity using an ear-electroencephalography (EEG) device, the ear-EEG device may need to be customized for a user's ear (e.g., possibly customized for the user's concha, ear canal, targus, etc.), and may need to be customized differently for different users, so that the electrodes placed on the ear-EEG device may remain in continuous contact with a user's body. Because an ear's size and shape vary from one user to another, and because a single user's ear size and shape, and size and shape of structures such as a user's ear canal, may change over time, even a customized ear-EEG device may fail to generate accurate measurements at times (or over time). In addition, a customized ear-EEG device may be expensive.

### SUMMARY

Embodiments/aspects described herein generally relate to a wearable electronic device for measuring biosignals of a user. The device may have more electrodes than are necessary to measure the biosignals for a given user, and the device may dynamically select a subset of the electrodes for measuring the biosignals for a given user at a given time. The device may dynamically select different subsets of the electrodes for measuring the biosignals for different users or at different times (e.g., depending on parameters of the user, how the device is being worn, ambient affects, etc.). In some cases, the embodiments/aspects described herein pertain to an earbud and/or a headset.

In one aspect, a wearable electronic device is described. The wearable electronic device may include a housing and an electronic carrier attached to the housing having a nonplanar surface. The wearable electronic device may include a set of electrodes including electrodes positioned at different locations on the nonplanar surface, a sensor circuit, and a switching circuit. The switching circuit is operable to electrically connect a number of different subsets of one or more electrodes in the set of electrodes to the sensor circuit.

In a second aspect, a wearable electronic device is described. The wearable electronic device may include a housing, a set of active electrodes, and a set of reference electrodes. The sets of active and reference electrodes are disposed in different positions or regions of the housing. The wearable electronic device includes a switching circuit configured to form one or more subsets of active electrodes each including at least one active electrode of the set of active electrodes, and one or more subsets of reference electrodes each including at least one reference electrode of the set of reference electrodes.

In a third aspect, an apparatus configured to measure a biosignal is described. The apparatus may include a first component configured to be worn on or in a first ear, and a second component configured to be worn on or in a second ear. The first component may include a set of active electrodes disposed in different regions of a housing of the first component, and the second component may include a set of reference electrodes disposed in different regions of a housing of the second component. The apparatus may include at least one switching circuit electrically coupled to the set of active electrodes and the set of reference electrodes. The apparatus may include at least one processor configured to form, using the at least one switching circuit, one or more subsets of active electrodes. The at least one processor may also be configured to form, using the at least one switching circuit, one or more subsets of reference electrodes. Each subset of active electrodes may include at least one active electrode of the set of active electrodes, and each subset of reference electrodes may include at least one reference electrode of the set of reference electrodes.

### BRIEF DESCRIPTION OF THE DRAWINGS

Reference will now be made to representative embodiments/aspects illustrated in the accompanying figures. It should be understood that the following descriptions are not intended to limit this disclosure to one included embodiment. To the contrary, the disclosure provided herein is intended to cover alternatives, modifications, and equivalents as may be included within the spirit and scope of the described embodiments, and as defined by the appended claims.

FIG. 1 depicts an example wearable electronic device, such as an earbud.

FIG. 2 depicts an example wearable electronic device, such as an earbud, as described herein, in accordance with some embodiments.

FIG. 3 depicts one view of a portion of an example wearable electronic device, such as an earbud, as described herein, in accordance with some embodiments.

FIG. 4 depicts one view of a portion of an example wearable electronic device, such as an earbud, as described herein, in accordance with some embodiments.

FIG. 5 depicts an example wearable electronic device, such as an earbud, and turning on or off a feature to measure a biosignal, as described herein, in accordance with some embodiments.

FIG. 6 depicts an electrical block diagram of a wearable electronic device, as described herein, in accordance with some embodiments.

FIGS. 7A-7C depict a number of patterns of electrodes on a housing of a wearable electronic device, as described herein, in accordance with some embodiments.

FIG. 8 depicts an electrical block diagram of a sensing circuit of a wearable electronic device, as described herein, in accordance with some embodiments.

FIG. 9 depicts an example of selection of one or more active electrodes, as described herein, in accordance with some embodiments.

FIG. 10 depicts a wearable electronic device, such as a wired headset, as described herein, in accordance with some embodiments.

FIGS. 11A-11B depict example views of biosignals measured by electrodes, as described herein, in accordance with some embodiments.

The use of the same or similar reference numerals in different figures indicates similar, related, or identical items.

Additionally, it should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Reference will now be made in detail to representative embodiments/aspects illustrated in the accompanying drawings. It should be understood that the following description is not intended to limit the embodiments to one preferred embodiment. On the contrary, it is intended to cover alternatives, modifications, and equivalents as can be included within the spirit and scope of the described embodiments as defined by the appended claims.

Embodiments described herein include a wearable electronic device, such as an earbud, a pair of earbuds, and/or a wired headset. The wearable electronic device, as described herein, may include a housing and a number of active and reference electrodes on the housing. The number of active and reference electrodes may be configured to measure biosignals of a user of the wearable electronic device. The biosignals measured by the wearable electronic device may include, but are not limited to, an electroencephalography (EEG). In some embodiments, other biosignals may be measured, such as an electromyography (EMG), electrooculography (EOG), an electrocardiogram (ECG), a galvanic skin response (GSR), a blood volume pulse (BVP), etc.

Measurement of a biosignal, such as an EEG, with a wearable electronic device such as an earbud or wired headset that can be placed inside an ear canal or around an ear, may require that the active and reference electrodes remain in contact with the user's body at particular locations. However, each person may have a different shape and size of ear or ear canal, and in some embodiments, the shape or size of the ear or ear canal may change over time. As a result, the active and/or reference electrodes may fail to make proper contact with the body of the user and, in turn, may reduce the accuracy of the biosignal measurements.

In some embodiments, inaccuracies of the biosignal measurements may be reduced and/or avoided by dynamically selecting one or more active electrodes of the number of active electrodes and/or one or more reference electrodes of the number of active electrodes for measuring a biosignal. The number of active electrodes and/or number of reference electrodes may be discretely placed on the housing of the wearable electronic device. In other words, each active electrode and each reference electrode may be disposed in a different region of the housing. The number of active electrodes and/or number of reference electrodes may be connected, in some embodiments, using a flex (e.g., flexible) circuit substrate and/or micro axial cables. In some embodiments, the electrode carrier may be an earpiece (e.g., an in-ear piece, an out-ear piece, or an over-ear piece) and/or a wall of the housing. In some embodiments, there may be a set of micro axial cables or a set of flex circuit substrates.

In some embodiments, the one or more active electrodes and the one or more reference electrodes that are dynamically selected may be selected based on criteria including, but not limited to, an impedance level, noise (e.g., ambient noise, and/or electrode-skin interface noise, etc.), physical distance between an active electrode and a reference electrode, half-cell potentials, etc. The selection of the one or more active electrodes forming a subset of active electrodes of one or more subsets of active electrodes, and the one or more reference electrodes forming a subset of reference electrodes of one or more subsets of reference electrodes may be performed before a session for measuring a biosignal is started. In some embodiments, and by way of a non-limiting example, the subset of active and/or reference electrodes may be periodically updated to add, update, and/or remove an active and/or reference electrode from a subset of active electrodes and/or subset of reference electrodes that is used to measure the biosignal.

In some embodiments, the selection of the one or more active and/or reference electrodes may be performed using a machine-learning based model. The machine-learning model may be configured to select the one or more (and in some cases all) active electrodes of the subset of active electrodes and/or the one or more (and in some cases all) reference electrodes of the subset of reference electrodes based on a number of features including, but not limited to, an impedance level, noise (e.g., ambient noise, and/or electrode-skin interface noise, etc.), physical distance between an active electrode and a reference electrode, half-cell potentials, etc., based on input data obtained from one or more user, or user's own calibration data. The machine-learning model may be based on a multi-modal learning, supervised learning, and/or unsupervised learning approach.

In some embodiments, instead of selecting one or more active electrodes, and/or one or more reference electrodes, all active electrodes and/or all reference electrodes may be selected for measuring a biosignal. However, each electrode of the active electrodes and/or each electrode of the reference electrodes may be assigned a different weight. The weight assigned to each electrode of the active electrodes and/or reference electrodes may be determined based on a number of features including, but not limited to, an impedance level, noise (e.g., ambient noise, and/or electrode-skin interface noise, etc.), physical distance between an active electrode and a reference electrode, half-cell potential, and/or a placement of an electrode, and so on. Additionally, or alternatively, the weight assigned to each electrode may be determined based on input data obtained from one or more uses, or user's own calibration data.

In some embodiments, a biosignal measured using each active electrode of the active electrodes and/or each reference electrode of the reference electrodes may be an EEG signal, such as an EEG signal in a frequency band (e.g., an EEG signal in an alpha frequency band of 8 to 12 Hz). A magnitude level of a raw signal corresponding to an electrode pair including an active electrode and a reference electrode, in the alpha frequency band, generally increases when a user closes their eyes, and decreases when the user opens their eyes. The magnitude level of the raw signal may be measured as a difference between a voltage measured at the active electrode (V_active) and a voltage measured at the reference electrode (V_ref), e.g., (V_active−V_ref). Accordingly, based on the raw signal corresponding to each pair of an active electrode and a reference electrode, along with its respective applied weight, an optimized signal in the frequency domain corresponding to all pairs of active and reference electrodes may be obtained. By way of a non-limiting example, the optimized signal corresponding to all pairs of active and reference electrodes may be a single time-series signal generated according to a respective weight assigned to each active and reference electrode pair. A ratio of the optimized signal corresponding to all active and reference electrode pairs when a user's eyes are closed to the optimized signal corresponding to all active and reference electrode pairs when the user's eyes are open is desired to be more than one. Accordingly, based on performing various measurements, an appropriate weight for each active and reference electrode pair may be determined and assigned.

In some embodiments, the subset of the one or more active electrodes (or all active electrodes) and/or the subset of the one or more reference electrodes (or all reference electrodes) may be formed by a switching circuit that electrically couples one electrode with another electrode. The switching circuit may be controlled by a processor, which may be, for example, a microcontroller, a field-programmable gate array (FPGA), an application-specific integrated circuit (ASIC), a digital signal processor (DSP), etc. The processor may be communicatively coupled with one or more memories that store instructions which, when executed by the processor, cause the processor to execute an algorithm or perform instructions to dynamically select the one or more active and/or reference electrodes of the subsets of the active and/or reference electrodes, respectively, as described herein. Additionally, or alternatively, the stored instructions, which when executed by the processor, cause the processor to perform measurements using all active electrodes and/or all reference electrodes, and determine and/or assign a respective weight value to each active and/or reference electrodes, as described herein.

As described herein, each user may have a different shape and size of an ear canal. Accordingly, an active electrode having one predetermined position on the housing may produce a signal of higher strength in one situation (e.g., for one user or at a particular time) but may not produce a good quality signal in another situation (e.g., for a different user or at a different time). As a result, a customized wearable electronic device in which the electrodes are placed at particular locations for a particular user, to obtain a good quality measurement from the particular user, may be useful. However, such a customized device may be expensive. A solution to this problem, as described in the present disclosure, is to place a number of active electrodes on a surface of a housing of a wearable electronic device, and to dynamically select a subset of the electrodes that is best able to measure the biosignal for a given user, at a given time, and possibly under a given set of other conditions (e.g., ambient conditions). A switching circuit may then be used to electrically couple the selected subset(s) of electrodes. Non-selected electrodes may be disabled, thus reducing the energy consumption of the wearable electronic device and improving the accuracy of the measurement.

In some embodiments, and by way of a non-limiting example, a number of active electrodes may be disposed in an ear tip region of the wearable electronic device, such as an earbud, and a number of reference electrodes may be disposed on the shell or region of the earbud which generally stays outside of an ear canal. As described herein, one or more active electrodes may be selected, based on an algorithm, such as a machine-learning model, according to criteria for selection of active electrodes. The selected electrodes may then be electrically coupled to the same channels using at least one switching circuit. By way of a non-limiting example, the electrodes may be placed in a number of different patterns or may be selected to form a pattern or a number of different patterns.

In some embodiments, a number of active electrodes may have a circular shape, such as a dot shape. The number of active electrodes may be placed around one or more concentric ring shapes, for example. The number of active electrodes may in some embodiments be disposed in one or more concentric rings at some distance apart. The distances between different pairs of adjacent electrodes may be uniform or different. The number of concentric rings in which the active electrodes are disposed may vary, and the spacings between different pairs of adjacent concentric rings may be the same or may vary. One or more active electrodes from one or more concentric rings may be selected, according to criteria described herein, forming a subset of active electrodes.

In some embodiments, a number of active electrodes may have a hybrid shape. In other words, the shape of an electrode may include a part circular, a part oval, a part square, and/or an undefined shape.

In some embodiments, a particular electrode ring of a number of active ring-shaped electrodes may be selected. In some embodiments, an active electrode may be of an oval shape, and thus may have a larger contact area with the skin of the user.

In some embodiments, a flex and/or a micro axial cable connecting a plurality of active electrodes and/or reference electrodes may have a variety of geometries connecting the active electrodes. For example, the flex and/or the micro axial cable may be shaped as a spine, an octopus, and/or a mesh, etc. The design selected for the flex and/or the micro axial cable may depend on the size of the wearable electronic device, for example. The flex and/or micro axial cable may be embedded inside the housing of the wearable electronic device, in some embodiments.

In some embodiments, in addition to the number of active and/or reference electrodes, there may be at least one ground (GND) connection. In some embodiments, the measurement may be started when a user touches or taps on a reference electrode of a number of reference electrodes. In some embodiments, there may be a single reference electrode which may be disposed on a shell occupying a larger area. The single reference node may be of any shape, such as a circle, an oval, and/or a triangle, etc.

In some embodiments, the wearable electronic device may also include a battery and amplifier or another suitable power supply. A voltage potential drop between the one or more active electrodes and corresponding one or more reference electrodes, which is an analog signal, may be amplified using an amplifier. The positive and negative battery terminals are connected to an amplifier that may provide an electrical signal (as a voltage and/or a current) as an input to one or more active electrodes and/or one or more reference electrodes.

In some embodiments, the amplified or non-amplified analog signal may then be converted to a digital signal using an analog-to-digital converter. The digital signal may be stored in a memory of the wearable electronic device. In some embodiments, the digital signal from the measurement may be transmitted to a user equipment, such as a mobile phone, using a wireless communication protocol, such as Bluetooth, and/or Wi-Fi, etc. In some embodiments, the measurements may be transmitted to a server using any radio access technology, such as 3G, 4G, 5G, 6G, Wi-Fi, WiMax, etc., and/or wireline technology. The server may store the measurement as historic data, which may be accessed by the user and/or another person given permission to access the measurements. The measurements of the biosignal, may thus be used to inform the user for various biosignal-driven use cases, such as a sleep monitoring or other anomalies, such as seizures.

In some embodiments, a filter such as a low-pass filter, and/or a bandpass filter may also be used to reduce noise level.

In some embodiments, it may be desirable that the distance between an active electrode and a corresponding reference electrode be as large as possible. Accordingly, the active electrodes and the reference electrodes may be disposed on different earbuds. For example, active electrodes may be placed on a right earbud and reference electrodes may be placed on a left earbud, or vice versa. At least one processor, and at least one switching circuit, located in any one of the earbuds may then form a subset of active electrodes and a subset of reference electrodes for measuring of the biosignal, as described in the present disclosure.

In some embodiments, the wearable electronic device may be a wired headset, and the active and reference electrodes may be on different sides of the headset. For example, active electrodes may be on a right side of the headset and reference electrodes may be on a left side of the headset. The active and reference electrodes may be electrically and/or communicatively coupled with a wire, a micro axial cable, and/or a flex substrate.

The foregoing and other embodiments, and various alternatives thereof and variations thereto, are discussed herein with reference to the drawings for purposes of explanation, and to facilitate an understanding of various configurations and constructions of a system, such as described herein. However, it will be apparent to one skilled in the art that some of the specific details presented herein may not be required in order to practice a particular described embodiment, or an equivalent thereof.

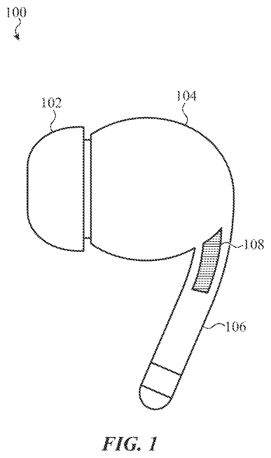

FIG. 1 depicts an example wearable electronic device, such as an earbud. As shown in FIG. 1, an earbud 100 of a left or a right ear may include a tip 102, an earpiece 104, and a stem 106. The tip 102 may be made of any material such as plastic, rubber, conductive material, silicone, or a mixture of these. In some embodiments, the housing underneath the tip 102 may be made of any of a metal, ceramic, glass, plastic material, or a mixture of these. The earpiece 104 and/or the stem 106 may also be made of any of a metal, ceramic, glass, plastic material, or a mixture of these. The hollow portion of the tip 102, the earpiece 104, and/or the stem 106 may form a housing of the earbud 100.

The housing of the earbud may include a circuit, which may further include, but is not limited to, one or more speakers, one or more microphones, a battery or another suitable power supply, a charging circuit for charging the battery or the other suitable power supply, an optical sensor, one or more motion accelerometers, a gyroscope, a communication circuitry, a processor, and/or a memory, etc.

In some embodiments, a shaded portion 108 shown in FIG. 1 may act as an input system to receive various commands from a user. The input system may include one or more of accelerometers, touch sensors, force sensors, pressure sensors, etc. A squeeze, a single tap, and/or a double tap on the shaded portion 108, for example, may be received as an input from a user by the input system, and may be interpreted as various commands, such as play music, answer a call, disconnect a call, increase/decrease volume, etc., depending on how the user has set up the earbud.

In some embodiments, an accelerometer may be used to determine an orientation of the earbud and automatically start or stop various functions. In some embodiments, upon detecting an earbud position that corresponds to the earbud being in an ear of a user, music may be automatically played, and/or another function may be automatically started.

In some embodiments, the communication circuitry may communicate with another user equipment (UE), for example, a phone of the user, using a communication protocol such as, Bluetooth, Wi-Fi, and/or Near Field Communication (NFC), etc. The communication circuitry may transmit and/or receive data from the other UE using the communication circuitry.

The processor may be a microprocessor, a microcontroller, a digital signal processor (DSP), an application-specific integrated circuit (ASIC), a field-programmable gate array (FPGA), etc. The memory may include read-only memory (ROM), random-access memory, and/or static random-access memory (SRAM), etc. The memory may store instructions corresponding to an algorithm and/or a machine-learning model to perform various functions as configured.

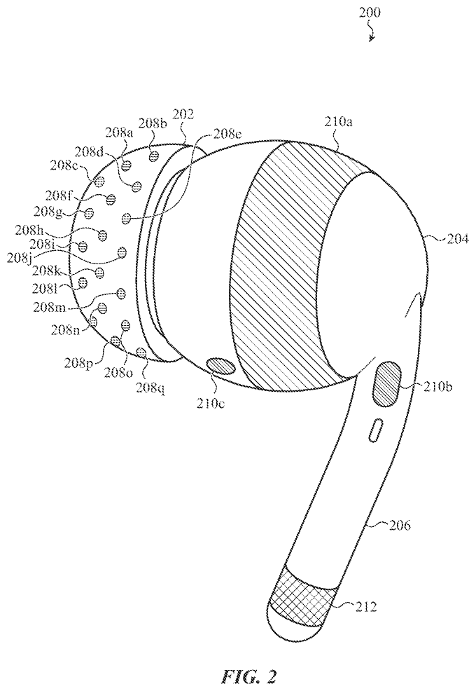

The earbud 100 may be used to measure a biosignal, for example, an electroencephalogram (EEG), for measuring brain activity. As described earlier, a currently available earbud to measure the EEG may include an active electrode on the tip 102 and a reference electrode on the earpiece 104 and/or the stem 106. Since the location or contact point for the active electrode to measure electrical activity of a brain of a user remains fixed, and each person has a different shape and size of an ear canal, the measured electrical activity for the EEG test may be not accurate, and/or may need an earbud customized according to the shape and size of an ear canal of the user. However, an earbud, as described herein using FIGS. 2-6, 7A-7C, and 8-10 according to some embodiments, may solve the problem of a customized earbud and/or inaccurate EEG measurement.

FIG. 2 depicts an example wearable electronic device, such as an earbud, as described herein, in accordance with some embodiments. The earbud 200 shown in FIG. 2 may include a tip 202, an earpiece 204, and a stem 206, similar to the earbud 100 shown in FIG. 1.

The tip 202 may be made of any material such as plastic, rubber, conductive material, silicone, or a mixture of these. In some embodiments, the housing underneath the tip 202 may be made of any of a metal, ceramic, glass, plastic material, or a mixture of these. The earpiece 204 and/or the stem 206 may also be made of any of a metal, ceramic, glass, plastic material, or a mixture of these. The hollow portion of the tip 202, the earpiece 204, and/or the stem 206 may form a housing of the earbud 200.

The housing of the earbud may include a circuit which may further include, for example, one or more speakers, one or more microphones, a battery or another suitable power supply, a charging circuit for charging the battery or the other suitable power supply, an optical sensor, one or more motion accelerometers, a gyroscope, communication circuitry, a processor, and/or a memory, etc., which may perform similar functions as described herein with reference to the earbud 100 shown in FIG. 1.

As shown in FIG. 2, a set of electrodes including a number of active electrodes 208a-208q are disposed on the tip 202 of the earbud 200. By way of a non-limiting example, the number of active electrodes 208a-208q may be on the surface of the tip 202 and/or embedded inside the material used to form the tip 202.

In some embodiments, the tip 202 may be made of an electrically conductive material. In some embodiments, the tip 202 may be made of an electrically conductive material when an active electrode is embedded inside the material used for the tip 202. In some embodiments, only areas where the active electrode is embedded inside the material used to form the tip 202 may be made of electrically conductive material.

In some embodiments, the set of electrodes may also include a number of reference electrodes 210a-210c disposed on the earpiece 204 of the earbud 200. In FIG. 2, three reference electrodes are shown, but there may be fewer than or more than three reference electrodes. In some embodiments, there may be a single reference electrode.

In some embodiments, and by way of a non-limiting example, the active electrodes 208a-208q and the reference electrodes 210a-210c may receive an electrical signal (as a voltage and/or a current) as an input from a battery or another suitable power supply. In some embodiments, the electrical input signal to an active electrode and/or a reference electrode may be amplified using an amplifier.

In FIG. 2, each of the number of active electrodes 208a-208q is shown to have a circular shape, such as a dot shape. The number of active electrodes may be placed around one or more concentric ring shapes, for example. However, as described herein, the active electrodes 208a-208q may have any suitable shape or size. The reference electrode 210a has a ring shape around the earpiece 204. The shape of the reference electrode 210a may be configured, for example, as shown in FIG. 2, such that it makes it convenient for a user to provide an input using a touch, a squeeze, and/or one or more taps, etc. The one or more reference electrodes 210a-210c may also be interspersed with one or more accelerometers and/or one or more force sensors to receive input from the user as described herein. Accordingly, a single tap in the area where the reference electrode 210a is shown, for example, may be interpreted as a command to start a measurement of a biosignal, for example, an EEG.

For measuring the EEG, one or more active electrodes or the number of active electrodes 208a-208q may be selected as a subset of active electrodes. Similarly, one or more reference electrodes of the number of reference electrodes 210a-210c may also be selected as a subset of reference electrodes. The one or more active electrodes of the subset of active electrodes may be connected and/or coupled using an electrode carrier on the housing of the earbud 200. As shown in FIG. 2, the housing of the earbud 200 has a nonplanar surface, and the electrode carrier may be a flex substrate or a flexible circuit substrate, a flex circuit, and/or a micro axial cable. In some embodiments, the electrode carrier may be an earpiece (e.g., an in-ear piece, an out-ear piece, or an over-earpiece). In some embodiments, the electrode carrier may be a wall of the housing, such as the nonplanar surface of the housing of the earbud 200.

In some embodiments, a ground electrode (GND) 212 may be placed on the housing of the wearable electronic device 200, as shown in FIG. 2. The GND 212 may be used to prevent noise from interfering with the small biopotential signals of interest.

In some embodiments, a switching circuit, as described herein, may electrically connect the one or more active electrodes (or all active electrodes) of the subset and/or a number of such subsets, which thus forms a sensor circuit. By way of a non-limiting example, the number of such subsets may include a first subset of one or more (or all) active electrodes, and second subset of one or more (or all) reference electrodes. The switching circuit may be controlled by the processor, in a first mode, according to an algorithm and/or a machine-learning model that is configured to select one or more active electrodes (or all active electrodes) based on features, including, but not limited to, an impedance level, noise (e.g., ambient noise, and/or electrode-skin interface noise, etc.), physical distance between an active electrode and a reference electrode, half-cell potentials, etc. In other words, in the first mode, an impedance between an active electrode (“a first electrode”) of a set of active electrodes and a reference electrode (“a second electrode”) of a set of reference electrodes may be measured, along with other parameters. In some embodiments, a physical distance between the first electrode and the second electrode may be known or calculated. The switching circuit and/or the sensor circuit may be controlled by the processor, in a second mode, to measure a biosignal, for example, an electrical activity in brain area. In other words, in the second mode, a voltage difference between the selected first and second electrodes may be measured for measuring the biosignal. By way of a non-limiting example, the biosignal may be an optimized biosignal corresponding to the first subset of active electrodes, and/or an optimized biosignal corresponding to the second subset of active electrodes generated by applying a respective weight to each active electrode of the first subset of active electrodes, and/or each reference electrode of the second subset of reference electrodes, respectively.

In some embodiments, the electrical activity measured by the sensor circuit may be further analyzed by the processor, and/or may be communicated to another user equipment (UE) and/or a server using the communication circuit mentioned herein. In some embodiments, the user may be sent a notification in accordance with the analyzed electrical activity from the measurement. The notification may be sent as an auditory signal, a visual signal, haptic feedback, an email and/or a text, etc.

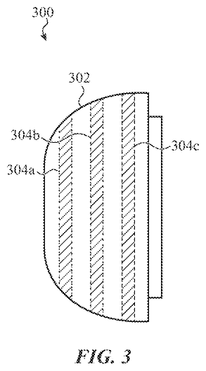

FIG. 3 depicts one view of a portion of an example wearable electronic device, such as an earbud, as described herein, in accordance with some embodiments. The portion of the earbud 300 shown in FIG. 3 is the tip 302 of the earbud, which has active electrodes 304a-304c. Each active electrode of the active electrodes 304a-304c on the tip 302 may be ring-shaped or shaped the same as (or differently from) the shape of an active electrode shown in FIG. 2. One or more active electrodes 304a-304c may be selected for measuring a biosignal, as described herein, in accordance with some embodiments.

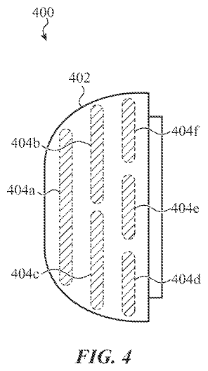

FIG. 4 depicts one view of a portion of an example wearable electronic device, such as an earbud, as described herein, in accordance with some embodiments. A portion of the earbud 400 shown in FIG. 4 illustrates a tip 402 of the earbud, which has active electrodes 404a-404f. Each active electrode of the active electrodes 404a-404f on the tip 402 may be of an oval shape, for example. Each active electrode of the active electrodes 404a-404f may be of the same size of a different size. One or more active electrodes 404a-404f may be selected for measuring a biosignal, as described herein, in accordance with some embodiments. In alternative embodiments, the electrodes 404a-404f may be rectangular-shaped or have other shapes. The electrodes 404a-404f may each have the same shape and size or may have different shapes or sizes.

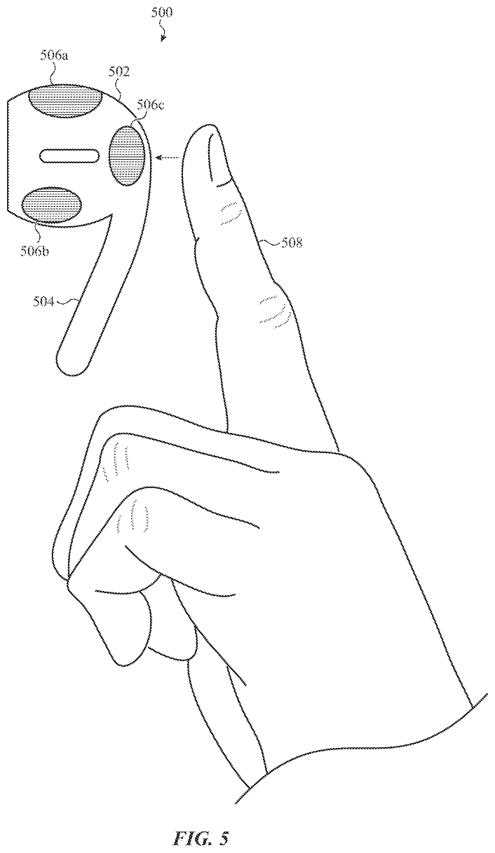

FIG. 5 depicts an example wearable electronic device, such as an earbud, and turning on or off a feature to measure a biosignal, as described herein, in accordance with some embodiments. The earbud 500 shown in FIG. 5 includes an earpiece 502 and a stem 504. One or more reference electrodes 506a-506c may be disposed on the earpiece 504. Each reference electrode of the one or more reference electrodes 506a-506c may be of an oval shape, for example. The reference electrodes may, however, be of any shape or size.

In some embodiments, a reference electrode may also be used to start or stop measurement of a biosignal. The reference electrode may also include or be coupled with a force sensor and/or accelerometer to receive an input from a user. The input from the user may be a tap, a touch, and/or a squeeze, etc., as shown in FIG. 5 as 508.

In some embodiments, the input from the user may be an audio input, which may be received at a microphone of the earbud. In some embodiments, an optical sensor in the earbud may also be configured to receive an input from the user to start and/or stop measurement of the biosignal.

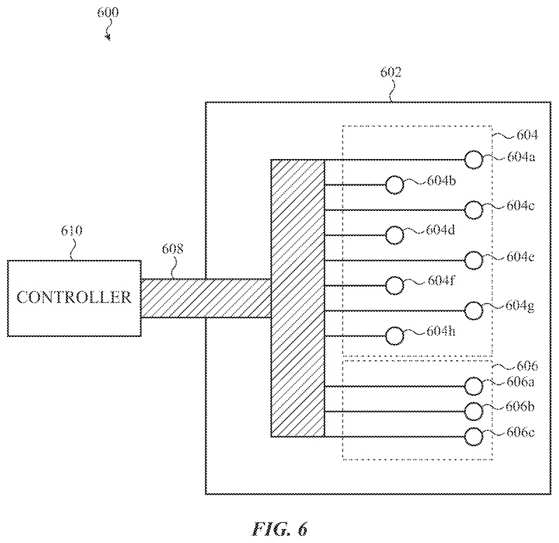

FIG. 6 depicts an electrical block diagram of a wearable electronic device 600, as described herein, in accordance with some embodiments. The electrical block diagram 600 includes a set of electrodes 602 including a number of active electrodes 604 and a number of reference electrodes 606. The number of active electrodes 604 may include active electrodes 604a-604h. The number of reference electrodes 606 may include reference electrodes 606a-606c. The active electrodes 604a-604h and the reference electrodes 606a-606c may be carried by an electrode carrier 608, which may be a flex and/or a set of microaxial cables and may be electrically coupled to a controller 610.

In some embodiments, the controller 610 may include a processor, a microcontroller, a DSP, an FPGA, and/or an ASIC, etc. The controller 610 may select one or more active electrodes 604a-604h to form a subset of active electrodes of a number of subsets of active electrodes. The one or more active electrodes 604a-604h may be selected based on criteria described herein, such as, including, but not limited to, an impedance level, noise (e.g., ambient noise, and/or electrode-skin interface noise, etc.), physical distance between an active electrode and a reference electrode, half-cell potentials, etc. Similarly, the controller 610 may select one or more reference electrodes 606a-606c to form a subset of reference electrodes of a number of subsets of reference electrodes. The controller 610 may then perform a measurement test using the selected subsets of active and reference electrodes. In one example, the measurement may be for measuring an EEG.

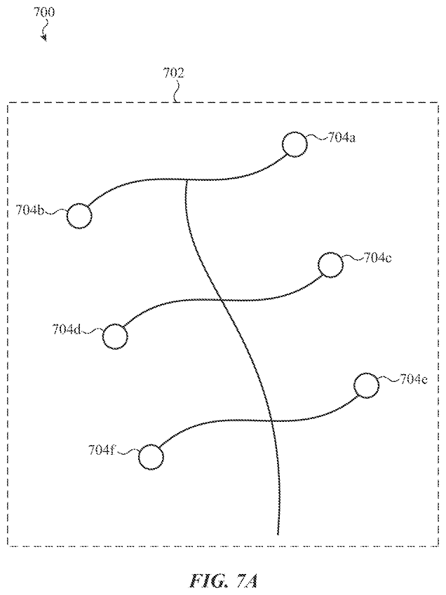

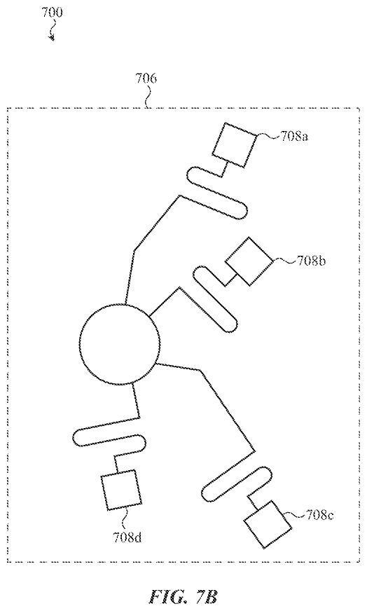

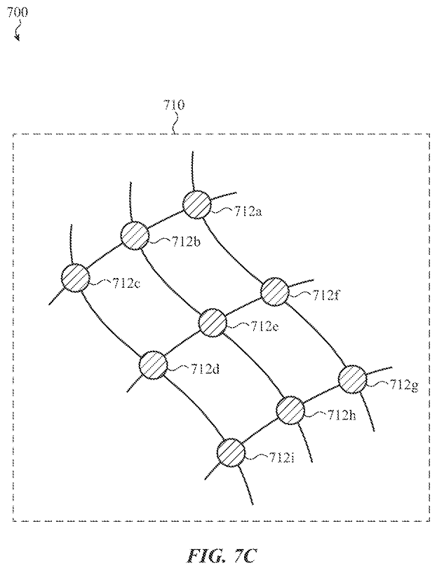

FIGS. 7A-7C depict a number of patterns of electrodes on a housing of a wearable electronic device, as described herein, in accordance with some embodiments. As shown in FIGS. 7A-7C, a block diagram 700 in each figure may represent a view of electrodes, such as a number of active electrodes arranged in a particular pattern. For example, in FIG. 7A, a view 702 is shown in which active electrodes 704a-704f are arranged in a spine form using an electrode carrier (e.g., a flexible circuit substrate). In FIG. 7B, a view 706 is shown in which active electrodes 708a-708d are arranged in an octopus shape using an electrode carrier (e.g., a flexible circuit substrate). In FIG. 7C, a view 710 is shown in which active electrodes 712a-712i are arranged in a mesh network using an electrode carrier (e.g., a flexible circuit substrate). A particular form or shape in which the active electrodes may be arranged is not limited to the ones described herein. The form or shape may be selected based on the size of an earbud and available space for routing of a flex substrate, flexible circuit substrate, and/or a set of one or more micro axial cables.

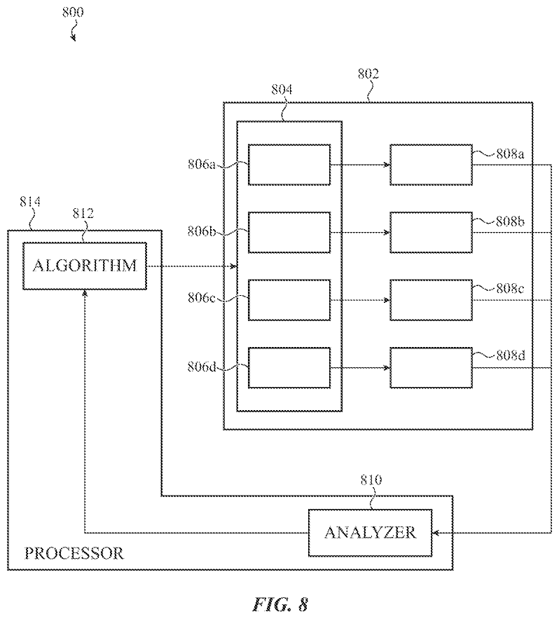

FIG. 8 depicts an electrical block diagram of a sensing circuit of a wearable electronic device 800, as described herein, in accordance with some embodiments. The sensing circuit 802 may include a switching circuit 804 including one or more switches 806a-806d. Each of the one or more switches 806a-806d may be connected to an active electrode and/or a reference electrode of a number of active and/or reference electrodes 808a-808d. A processor 814, which may be similar to the controller 610, may include and/or execute an algorithm 812 to select one or more active electrodes to form a subset of active electrodes of a number of subsets of active electrodes, and to select one or more reference electrodes to form a subset of reference electrodes of one or more subsets of reference electrodes. The processor 814 may evaluate the criteria for selection of one or more active electrodes using an analyzer 810. The analyzer may evaluate criteria including, but not limited to, an impedance level, noise (e.g., ambient noise, and/or electrode-skin interface noise, etc.), physical distance between an active electrode and a reference electrode, half-cell potentials, etc. The processor 814, using the algorithm 812, may initiate measurement of a biosignal using the selected subset of active electrodes and the selected subset of reference electrodes. In some cases, the electrodes of a subset of electrodes may be temporarily ganged, and a measurement of the biosignal may be obtained as a voltage between a subset of active electrodes and a subset of reference electrodes. In some cases, a biosignal may be measured using different pairs of subsets of electrodes (e.g., using a first subset of active electrodes and a first subset of reference electrodes; then using a second subset of active electrodes and the first subset of reference electrodes; then using the first subset of active electrodes and a second subset of active electrodes; and so on). In some cases, a measurement of a biosignal may be obtained as a voltage between a pair of active and reference electrodes selected from a subset of active electrodes and a subset of reference electrodes. In some cases, measurements of a biosignal may be obtained, sequentially, using different pairs of active and reference electrodes selected, respectively, from a subset of active electrodes and a subset of reference electrodes. Each subset of electrodes may include one or multiple electrodes.

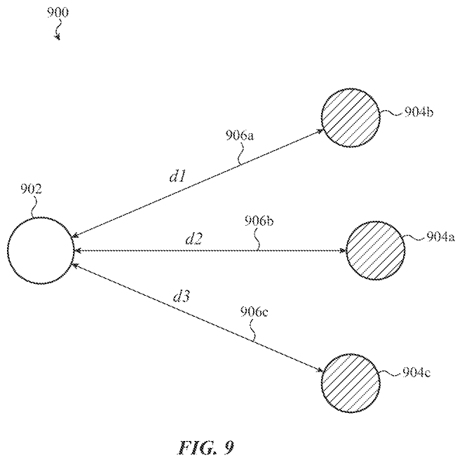

FIG. 9 depicts an example of selection of one or more active electrodes 900, as described herein, in accordance with some embodiments. As shown in FIG. 9, a reference electrode 902 may be d1 906a, d2 906b, and d3 906c distance away from active electrodes 904b, 904a, and 904c, respectively. In some embodiments, an impedance between an active electrode and the reference electrode may depend on a distance between them. The impedance may increase with an increased distance between the active and reference electrodes. A higher impedance between the active and reference electrodes may produce a more accurate measurement of an EEG. Accordingly, an active electrode which is farthest from a reference electrode may be selected for a subset of active electrodes.

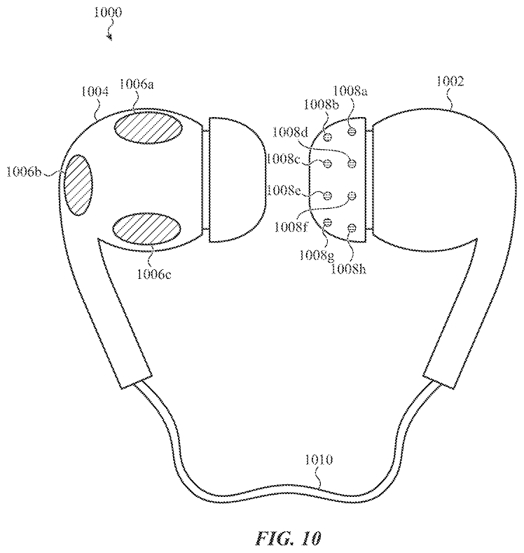

FIG. 10 depicts a wearable electronic device, such as a set of earbuds, as described herein, in accordance with some embodiments. A set of earbuds 1000 may include a first earbud 1002 and a second earbud 1004. On the first earbud 1002, there may be a number of active electrodes (or a set of active electrodes) 1008a-1008h, and on the second earbud 1004, there may be a number of reference electrodes (or a set of reference electrodes) 1006a-1006c. The first and the second earbuds 1002 and 1004 may be electrically and/or communicatively coupled with each other using a cable 1010. In some embodiments, at least one switching circuit (such as shown in FIG. 6) may be electrically coupled to the number of active electrodes and the number of reference electrodes.

In some embodiments, the wearable electronic device shown in FIG. 10 may alternatively be a set of earphone pieces or a headset. For example, the first earbud 1002 may be replaced with a first earphone piece (or first earpiece) of the set of earphone pieces (or headset), and the second earbud 1004 may be replaced with a second earphone piece (or second earpiece) of the set of earphone pieces (or headset). The first earphone piece and second earphone piece may be electrically and/or communicatively coupled with each other using a cable 1010. In some embodiments, the cable 1010 may be replaced with a headband that physically, electrically, and/or communicatively couples the first and the second earphone pieces.

Various features described herein with reference to FIG. 2 through FIG. 9 may be incorporated into the wearable electronic device 1000. Accordingly, those embodiments are not described here again for brevity. The wearable electronic device 1000 may provide more distance between the active electrodes and the reference electrodes because they are disposed on different earbuds. In some embodiments, an electromagnetic field change between the active and reference electrodes may be used to measure a biosignal.

In some embodiments, the wearable electronic device shown in FIG. 10 may alternatively be a pair of glasses, and active and reference electrodes may be placed in temple areas of the pair of glasses. For example, active and reference electrodes may be respectively positioned on a first stem and a second stem of the pair of glasses. The active and reference electrodes may be electrically coupled or decoupled as described herein in accordance with some embodiments.

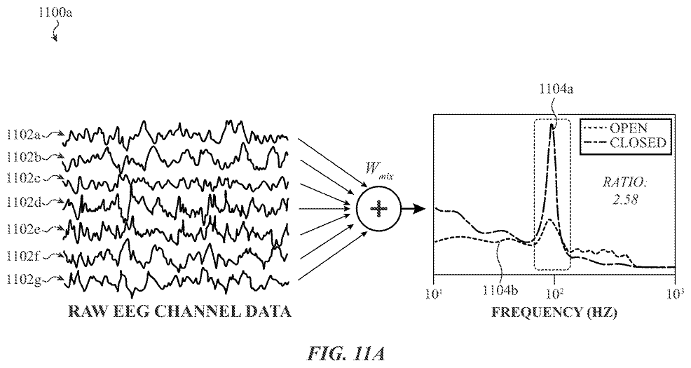

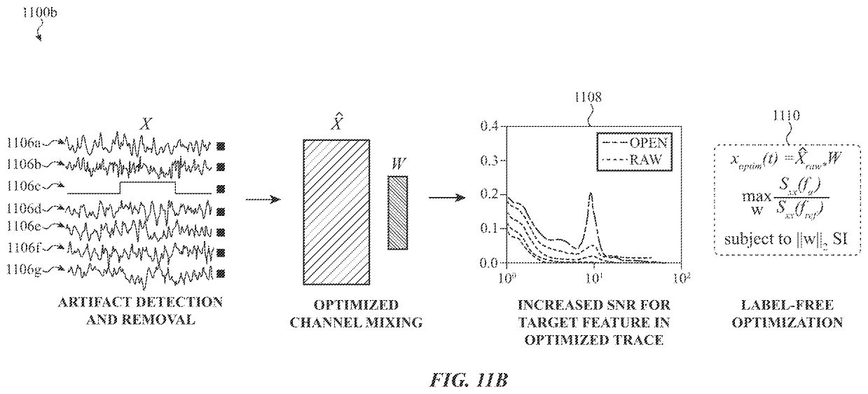

FIGS. 11A-11B depict example views of biosignals measured by electrodes, as described herein, in accordance with some embodiments. As shown in a view 1100a of FIG. 11A, biosignals 1102a-g may be measured by electrodes of the wearable electronic device 1000. The biosignals 1102a-g may include biosignals measured by all or a subset of active electrodes, and/or all or a subset of reference electrodes. The biosignals 1102a-g are time-series signals, with each biosignal 1102a-g measured by a different pair (or subset) of an active electrode and a reference electrode. Since each active electrode and/or each reference electrode may be assigned a different weight, or each pair of active and reference electrodes may be assigned a different weight, according to criteria including, but not limited to, an impedance level, noise (e.g., ambient noise, and/or electrode-skin interface noise, etc.), physical distance between an active electrode and a reference electrode, half-cell potential, and/or placement of an electrode, and so on, an optimized signal (i.e., a first biosignal) corresponding to a weighted sum of biosignals for all pairs of active electrodes and reference electrodes when eyes are closed may be generated, which is shown in FIG. 11A as optimized signal 1104a, and an optimized signal (e.g., a second biosignal) corresponding to a weighted sum of bio signals for all pairs of active electrodes and reference electrodes when eyes are open may be generated, which is shown in FIG. 11A as optimized signal 1104b. Each of the optimized signals 1104a and 1104b is presented for an alpha band (8-12 Hz), and each of the optimized signals 1104a and 1104b may be generated by processing a single time-series signal from pairs of an active electrode and/a reference electrode when eyes are closed and eyes are open, respectively, using a Fast Fourier transform (FFT) algorithm. Using the FFT algorithm, various frequency components of time-varying signals in accordance with a respective weight assigned to each active electrode and/or each reference electrode and/or each pair of active and reference electrodes may be identified. The resultant optimized signals corresponding to when eyes are closed and when eyes are open are shown in FIG. 11A as 1104a and 1104b, respectively.

In some cases, it may be desirable that a ratio of the optimized signal 1104a, corresponding to when eyes are closed, at its peak value for an amount energy to the optimized reference signal 1104b, corresponding to when the eyes are open, at its peak value for an amount of energy, is greater than 1. Accordingly, a respective weight for each active and/or reference electrode, or each pair of active and reference electrodes, may be determined (e.g., selected and then adjusted, as needed) and assigned to achieve this goal.

As shown in a view 1100b of FIG. 11B, biosignals 1106a-g each may represent biosignals as measured by electrodes of the wearable electronic device 1000. The biosignals 1106a-g may include biosignals measured by all or a subset of active electrodes, and/or all or a subset of reference electrodes. However, a biosignal 1106c may be a result of some artifact or poor contact with skin of the user. Upon detection of the artifact related to the biosignal 1106c, the artifact may be removed by giving zero or a minimum weight to an electrode detecting the biosignal 1106c, or giving zero or a minimum weight to a time window (or time windows) in which the electrode is not generating a biosignal 1106c containing useful information. An optimized signal thus generated is shown along with (raw or as measured) biosignals as 1108. An example formular to generate the optimized signal is shown in FIG. 11B as 1110. However, the optimized signal may be generated using other formulas or algorithms as well.

In an example algorithm Xₒₚₜᵢₘ(t)=X_{raw}*W, shown in FIG. 11B as 1110, W may represent weights given to each raw signals X_{raw} to generate the optimized signal. Further, W may be selected such that a ratio of the energy of an optimized signal in a certain frequency band, e.g., a frequency band fₐ, generated from pairs of an active electrode and a reference electrode to the energy of an optimized signal in a certain reference frequency band, e.g., a frequency band f_{ref}, generated from pairs of an active electrode and a reference electrode, is maximized.

As used herein, the phrase “at least one of” preceding a series of items, with the term “and” or “or” to separate any of the items, modifies the list as a whole, rather than each member of the list. The phrase “at least one of” does not require selection of at least one of each item listed; rather, the phrase allows a meaning that includes at a minimum one of any of the items, and/or at a minimum one of any combination of the items, and/or at a minimum one of each of the items. By way of example, the phrases “at least one of A, B, and C” or “at least one of A, B, or C” each refer to only A, only B, or only C; any combination of A, B, and C; and/or one or more of each of A, B, and C. Similarly, it may be appreciated that an order of elements presented for a conjunctive or disjunctive list provided herein should not be construed as limiting the disclosure to only that order provided.

One may appreciate that although many embodiments are disclosed herein, that the operations and steps presented with respect to methods and techniques described herein are meant as exemplary and accordingly are not exhaustive. One may further appreciate that alternate step order or fewer or additional operations may be required or desired for particular embodiments.

Although the disclosure herein is described in terms of various exemplary embodiments and implementations, it should be understood that the various features, aspects, and functionality described in one or more of the individual embodiments are not limited in their applicability to the particular embodiment with which they are described, but instead can be applied, alone or in various combinations, to one or more of some embodiments, whether or not such embodiments are described and whether or not such features are presented as being a part of a described embodiment. Thus, the breadth and scope of the present description should not be limited by any of the exemplary embodiments described herein but is instead defined by the claims herein presented.

What is claimed is:

1. A wearable electronic device, comprising:
a housing;
an electrode carrier attached to the housing and having a nonplanar surface;
a set of electrodes including electrodes positioned at different locations on the nonplanar surface;
a sensor circuit; and
a switching circuit operable to electrically connect a number of different subsets of one or more electrodes in the set of electrodes to the sensor circuit.

2. The wearable electronic device of claim 1, further comprising:
a processor in communication with the sensor circuit and the switching circuit.

3. The wearable electronic device of claim 2, wherein the processor is configured to:
in a first mode, control the switching circuit to sequentially connect the number of different subsets of the one or more electrodes to the sensor circuit via the switching circuit;
conduct one or more measurements for each of the number of different subsets of the one or more electrodes connected to the sensor circuit;
identify, at least partially in response to the one or more measurements for each of the number of different subsets of the one or more electrodes, a subset from the number of different subsets of the one or more electrodes for acquiring measurement of a biosignal;
in a second mode, control the switching circuit to connect the identified subset of the one or more electrodes to the sensor circuit, the one or more electrodes used for acquiring the measurement of the biosignal; and
operate the sensor circuit to obtain the measurement of the biosignal while the identified subset of the one or more electrodes for acquiring the measurement of the biosignal is connected to the sensor circuit.

4. The wearable electronic device of claim 3, wherein:
the number of different subsets includes a first subset of one or more active electrodes, and a second subset of one or more reference electrodes; and
an optimized biosignal corresponding to different pairs of an active electrode from the first subset of one or more active electrodes and a reference electrode from the second subset of one or more reference electrodes is generated by applying a respective weight to each active electrode of the first subset and each reference electrode of the second subset.

5. The wearable electronic device of claim 1, wherein the sensor circuit comprises a set of one or more reference electrodes disposed on an exterior surface of the housing.

6. The wearable electronic device of claim 1, wherein:
the electrode carrier includes a flexible circuit substrate; or
the electrode carrier includes an in-ear piece.

7. The wearable electronic device of claim 1, wherein the electrode carrier is a wall of the housing.

8. The wearable electronic device of claim 1, wherein the one or more electrodes in the set of electrodes includes at least an active electrode or a reference electrode, the active electrode or the reference electrode disposed on the electrode carrier.

9. A wearable electronic device, comprising:
a housing;
a set of active electrodes disposed in different regions of the housing;
a set of reference electrodes disposed in the different regions of the housing; and
a switching circuit configured to:
form one or more subsets of active electrodes each comprising at least one active electrode of the set of active electrodes, and
form one or more subsets of reference electrodes each comprising at least one reference electrode of the set of reference electrodes.

10. The wearable electronic device of claim 9, further comprising:
a processor configured to:
identify a subset of the one or more subsets of active electrodes for sensing a biosignal; and
identify a subset of the one or more subsets of reference electrodes for sensing the biosignal.

11. The wearable electronic device of claim 10, wherein identifying the subset of the one or more subsets of active electrodes or the subset of the one or more subsets of reference electrodes comprises:
selecting the at least one active electrode of the set of active electrodes based on an analysis of one or more of: impedances between a first electrode of the set of active electrodes and a second electrode of the set of reference electrodes, and physical distances between the first electrode and the second electrode.

12. The wearable electronic device of claim 10, wherein identifying the subset of the one or more subsets of active electrodes comprises:
selecting the at least one active electrode of the set of active electrodes based on a contact area with a body of a user for each of the set of active electrodes.

13. The wearable electronic device of claim 10, wherein the processor is further configured to:
control the switching circuit for acquiring measurements corresponding to the biosignal being sensed using the identified subset of the one or more subsets of active electrodes and the identified subset of the one or more subsets of reference electrodes.

14. The wearable electronic device of claim 10, wherein the biosignal is an electrical activity of a brain of a user.

15. The wearable electronic device of claim 9, wherein the wearable electronic device is a wireless earbud device or a wired headphone device.

16. The wearable electronic device of claim 9, wherein the housing comprises a first region and a second region, and the set of active electrodes is disposed in the first region and the set of reference electrodes is disposed in the second region.

17. The wearable electronic device of claim 9, wherein at least one electrode of the set of active electrodes has a ring shape, a circular shape, a dot shape, an oval shape, or a hybrid shape.

18. The wearable electronic device of claim 9, wherein an electrode of the set of active electrodes is coupled with another electrode of the set of active electrodes, or an electrode of the set of reference electrodes is electrically coupled with another electrode of the set of reference electrodes, using a set of micro axial cables or a flex.

19. An apparatus configured to measure a biosignal, the apparatus comprising:
a first component configured to be worn on or in a first ear, the first component comprising a set of active electrodes disposed in different regions of a housing of the first component;
a second component configured to be worn on or in a second ear, the second component comprising a set of reference electrodes disposed in different regions of a housing of the second component;
at least one switching circuit electrically coupled to the set of active electrodes and the set of reference electrodes; and
at least one processor configured to:
form, using the at least one switching circuit, one or more subsets of active electrodes each comprising at least one active electrode of the set of active electrodes, and
form, using the at least one switching circuit, one or more subsets of reference electrodes each comprising at least one reference electrode of the set of reference electrodes.

20. The apparatus of claim 19, wherein:
the at least one processor is further configured to,
identify a subset of the one or more subsets of active electrodes or a subset of the one or more subsets of reference electrodes based on an analysis of one or more of: impedances between first electrodes of the set of active electrodes and second electrodes of the set of reference electrodes; and
the biosignal is an electrical activity of a brain of a user.
